(19) United States
(12) Patent Application Publication
(10) Pub. No.: US 2026/0207488 A1
(43) Pub. Date: Jul. 23, 2026

(54) Liquid pharmaceutical composition containing flecainide and process for the preparation thereof

(71) Applicant: Colonis Pharma Ltd, London (GB)

(72) Inventors: Angelos KARATZAS, Koropi (GR); Argyro STAPPA, Koropi (GR); Konstantinos APOSTOLOU, Koropi (GR); Akis LOUKAS, Koropi (GR)

(21) Appl. No.: 19/141,552

(22) PCT Filed: Dec. 22, 2022
(86) PCT No.: PCT/EP2022/000114

§ 371 Date: Jun. 20, 2025

(51) Int. Cl. A61K 9/00; A61K 9/08; A61K 31/4458; A61K 47/12; A61K 47/26; A61K 47/46

(52) U.S. Cl. CPC A61K 9/0095; A61K 9/08; A61K 31/4458; A61K 47/12; A61K 47/26; A61K 47/46

(57) ABSTRACT

The present invention relates to a liquid pharmaceutical composition comprising a therapeutically effective quantity of flecainide or a pharmaceutically acceptable salt thereof, as an active ingredient, for administration in the pediatric population and adults who are unable to swallow a solid dosage form and a process for the preparation of said liquid pharmaceutical composition.

### TECHNICAL FIELD OF THE INVENTION

The present invention relates to a pharmaceutical composition for oral administration, and particularly to a liquid pharmaceutical composition comprising a therapeutically effective quantity of flecainide or a pharmaceutically acceptable salt thereof, as an active ingredient, for administration in the pediatric population and adults who are unable to swallow a solid dosage form. Furthermore, the present invention relates to a process for the preparation of said liquid pharmaceutical composition.

### BACKGROUND OF THE INVENTION

Flecainide is an antiarrhythmic agent used to treat tachyarrhythmias including atrial fibrillation, supraventricular tachycardia or ventricular tachycardia, particularly when conventional treatment agents fail.

Flecainide is commercially available as solid dosage form (tablets) and intravenous injection solutions. In absence of any oral liquid formulation available in the market, pharmacies generally follow a practice of preparing suspensions from available solid formulation by adding purified water. If required syrup can also be added to make the suspension palatable. The same practice is observed for Flecainide wherein the commercially available tablet formulation is crushed and mixed with water and syrup or some sweetener to prepare a palatable oral suspension for the patient.

Therefore, the solid dosage form must be compounded into a suspension when needed for infants and small children. Unfortunately, errors during preparation and dosing of such suspensions prepared from solid formulation have occasionally led to serious overdoses that resulted in cardiac emergencies and required immediate therapeutic intervention. Further, the stability of the suspensions obtained are very limited and cannot be stored for longer period.

Furthermore, there are patient populations that have difficulties swallowing solid dosage forms such as tablets and capsules. Children younger than 7-8 years old predominately prefer oral liquids over oral solids. Geriatric patients who are in old age or have suffered from certain illnesses, frequently have difficulty swallowing commonly prescribed oral solid dosage forms and therefore, also prefer oral liquids for drug administration.

U.S. Pat. No. 3,900,481 discloses Flecainide and its salts along with process for preparing the same. Flecainide Acetate is chemically known as N-(2-piperidinylmethyl)-2,5-bis(2,2,2-trifluoroethoxy)benzamide monoacetate.

Flecainide Acetate is a white to slightly off-white crystalline powder and is soluble in water and absolute alcohol with an aqueous solubility of about 48.0 mg/mL.

Flecainide Acetate falls under Class I of BCS (Biopharmaceutical Classification System) because of its high solubility and high permeability. However, due to the method of preparing suspension by crushing a tablet formulation, the resulting, final formulation will remain in suspension form rather than solution form because of the excipients present in the tablet formulation, which may or may not be soluble in water.

Therefore, there still exists the need to provide a liquid pharmaceutical formulation of flecainide acetate which exhibits an adequate release of flecainide acetate and a process for obtaining it in a good yield, which overcomes the related problems of the prior art and which is suitable for oral administration without any stability or dose uniformity issues.

### SUMMARY OF THE INVENTION

It is therefore, an object of the present invention to provide a storage-stable liquid pharmaceutical composition for oral administration comprising Flecainide or pharmaceutically acceptable salt thereof, as an active ingredient, which overcomes the deficiencies of the prior art.

It is another object of the present invention to provide a storage-stable liquid solution containing flecainide or a pharmaceutically acceptable salt thereof, as an active ingredient, which is safe and effective with sufficient self-life and good pharmacotechnical properties.

Moreover, it is another object of the present invention to provide a suitable process for the preparation of a storage-stable liquid pharmaceutical composition for oral administration containing flecainide or pharmaceutically acceptable salt thereof, as an active ingredient, which is effective and reproducible.

In accordance with the above objects of the present invention, a storage-stable liquid composition for oral administration is provided comprising a therapeutically effective amount of flecainide or a pharmaceutically acceptable salt thereof, as an active ingredient, wherein said active ingredient is from about 0.1 to about 3.0% w/v, wherein the weight/volume percentages are expressed as g/100 mL units, wherein said composition has a pH value below 4.0.

According to another embodiment of the present invention a storage-stable liquid composition for oral administration is provided comprising 0.5% Flecainide acetate, sucralose, a citrate buffering system which comprises citric acid, acetic acid or mixtures thereof, sodium benzoate in the range from 0.6 to 1.2 mg/mL and said composition is having pH value in the range from 2.8 to 3.2.

According to another embodiment of the present invention, a process for the preparation of a storage-stable liquid composition for oral administration comprising a therapeutically effective amount of flecainide or a pharmaceutically acceptable salt thereof, as an active ingredient, wherein said active ingredient is from about 0.1 to about 3.0% w/v, wherein the weight/volume percentages are expressed as g/100 mL units, is provided, wherein said process comprises the following steps:
(a) dissolving in purified water at least one of the excipients selected from buffering agents, and/or preservatives;
(b) adding the total amount of flecainide or salt thereof, to the resulting solution of step (a) and stirring for appropriate time until completely dissolved;
(c) optionally adding to the solution of step (b) at least one of the excipients selected from sweeteners and/or flavouring agents, and
(d) adjusting the pH of the final solution to a value below 4.0 with suitable hydrochloric acid or sodium hydroxide solution.

Further preferred embodiments of the present invention are defined in dependent claims 2 to 10 and 13 to 15. Other objects and advantages of the present invention will become apparent to those skilled in the art in view of the following detailed description.

### DETAILED DESCRIPTION OF THE INVENTION

For the purposes of the present invention, a liquid pharmaceutical composition for oral administration comprising flecainide or pharmaceutically acceptable salt thereof, as an active ingredient, is considered to be “stable” if said active ingredient precipitates less or more slowly than it does on its own and/or in known pharmaceutical compositions during storage.

An excipient is considered to be “incompatible” with said active ingredient or salt thereof if it promotes the degradation of said active ingredient, that is to say, if said active ingredient precipitates more or faster in the presence of said excipient when compared with the precipitation of said active ingredient on its own. The terms “incompatibility”, “compatible” and “compatibility” are defined accordingly.

The aim of the present invention was the development of liquid formulations containing a flecainide in order to achieve strong therapeutic effects and to ensure patient compliance and convenience in the treatment of cardiac arrhythmias including atrial fibrillation, supraventricular tachycardia and/or ventricular tachycardia. Under this scope, several different solutions were prepared and studied for their stability or incompatibility. In these studies, the formation of a solid precipitate was investigated during product storage, in order to assess the influence of conventionally used excipients like cosolvents, buffers, preservatives, sweeteners and flavouring agents.

It has been surprisingly found that the object of the present invention is achieved by adjusting the concentration of the active ingredient and the pH value of the mixture in the final solution, and by controlling the sequence of active ingredient and excipients addition, in order to prevent the formation of a precipitate and to improve the physicochemical stability of the active ingredient. Overcoming this technological hurdle enabled the preparation of a storage-stable composition containing effective amount of flecainide acetate.

Accordingly, in a first aspect the present invention provides a storage-stable liquid composition for oral administration comprising from about 0.1 to about 3.0% w/v flecainide acetate expressed as weight/volume units (g/100 mL). In the liquid composition of the present invention, flecainide can be used at different solvates or degrees of hydration, preferably as the acetate salt of flecainide.

In a preferred embodiment, the composition comprises about 0.5% w/v flecainide acetate. In addition to flecainide acetate the composition of the invention may comprise excipients suitable for oral liquid formulations and preferably co-solvents, solubilizers/surfactants, pH buffers or adjusting agents, preservatives, sweeteners and flavouring agents. In a preferred embodiment the composition contains, independently from one another: citric acid as buffer; sodium benzoate as preservative; sucralose as sweetener; cherry flavour as flavouring agent; sodium hydroxide or hydrochloric acid to adjust the pH value.

To achieve stability of the product, the pH value of the composition is preferably adjusted in the range below 4.0. It was found that outside this pH range, a precipitate may form during product storage regardless of the addition of solubilizers.

In the first basic studies on the present invention it was found that in the process of formulating a liquid solution containing flecainide acetate unexpected stability problems arise when the pH of the composition was above 7.0, wherein a precipitate was observed, revealing that the API is incompatible at pH value above 7.0. Best results in terms of absence of precipitate were observed in pH range below 4.0, with particularly good results in the pH range from 2.8 to 3.2. Also, it was noticed that the sequence of active ingredient and excipients addition at steps (a) and (b), seemed to have added robustness to the overall stability of the final product.

The liquid composition of the present invention is used to prepare oral solution. To this purpose, containers appropriate for oral use are filled with the liquid solution optionally sealing the containers with screw cap.

In a further aspect, the invention concerns the use of the liquid composition to treat a heart condition (e.g., cardiac arrhythmic) in a subject in need thereof.

The pharmaceutical compositions of the present invention may also contain one or more additional formulation ingredients selected from a wide variety of excipients. According to the desired properties of the composition, any number of ingredients may be selected, alone or in combination, based upon their known uses in preparation of liquid compositions for oral administration.

Such ingredients include, but are not limited to, co-solvents, buffers, sweeteners, flavouring agents, and preservatives.

The optional excipients must be compatible with Flecainide or salt thereof so that it does not interfere with it in the composition.

Moreover, any excipient may optionally be added to the above composition, provided that they are compatible with the active ingredient of the composition, in order to overcome problems associated with unfavorable pharmacotechnical characteristics of these substances, and in order to increase the self-life of the pharmaceutical product and provide a product exhibiting excellent stability, bioavailability and palatability.

The composition of the present invention may include further additives (alone or in a combination) such as acids, adjuvants, anticoagulants, antimicrobials, antiseptics, chelating agents, solubilizers, emollients, emulsifiers, flavor moisturizers, bufferants, pH control agents, stabilizers, suspending agents, sweeteners, thickening agents, surfactants, coloring agents, preservatives, etc.

Suitable sweeteners may be selected from sugars such as sucrose, lactose and glucose; cyclamate and salts thereof; saccharin and salts thereof; sucralose, aspartame and the like.

Flavouring agents may be selected from natural or synthetic flavours such as strawberry flavour, cherry flavour, orange flavour, green apple flavour, spearmint flavor, peppermint flavor and the like.

Solubilizers may be selected from sodium lauryl sulphate or complex forming agents such as cyclodextrins, ion exchange resins, crown ethers and the like.

One of the main objects of the present invention was to prepare a product with acceptable stability. For this reason the composition of the present invention was exposed to normal and accelerated stability studies according to the current ICH guidelines.

The results showed that the stability of the present invention was good (e.g. total impurities were not increased throughout normal and accelerated conditions).

The selection of appropriate materials (excipients, etc.) should be done carefully in order to avoid any incompatibility problems or non-compliance with EMA and FDA guidelines for inactive ingredients.

The following examples illustrate preferred embodiments in accordance with the present invention without limiting the scope or spirit of the invention:

### EXAMPLES

### Example 1: Compatibility Studies of Flecainide Acetate

An extensive compatibility study was carried out for the identification of potential drug—excipient interactions. The compatibility protocol was designed to include two different pH zones, an acidic pH zone with pH values pH≤4.0 and an alkaline pH zone pH>7.

Prior to evaluating the potential effects of inactive ingredients on the stability of flecainide acetate, its pH stability profile in the above specified pH areas was determined, directing thereafter the design of the compatibility studies. To this end, the compositions presented in Table 1 and 2 were placed at stressed heat conditions.

Drug-excipient interactions were monitored against related substances through stability indicating HPLC method.

### Example 1—Compositions 1-3 and Compositions 6-8 Containing 0.5% Flecainide Acetate for Establishing the pH-Stability Profile of Flecainide Acetate

TABLE 1
Composition 1-3 of the present invention.

| | Composition 1 | Composition 2 | Composition 3 |
|---|---|---|---|
| | Concentration | Concentration | Concentration |
| Ingredient | (mg/mL) | (mg/mL) | (mg/mL) |
| Flecainide acetate | 5.00 | 5.00 | 5.00 |
| Citric acid | — | 2.00 | — |
| Sodium citrate | — | 1.00 | — |
| Hydrochloric acid | q.s. to pH 3.5 | q.s. to pH 3.5 | q.s. to pH 4.0 |
| 1N Solution | | | |

Unless otherwise indicated, all steps in this procedure were carried out at room temperature.

The preparation of compositions 1 and 3 of Example 1 of the present invention was prepared according to the following process: Approximately 85% of the total volume of purified water was added in the compounding vessel and the total amount of Flecainide acetate was added under stirring and when complete dissolution was achieved additional purified water was added to the fill volume as required. The final pH value of the solution was measured with a calibrated pH-meter and, if necessary, it was adjusted to pH value according to Table 1 by using hydrochloric acid or sodium hydroxide solution. The results are presented below (see Table 3).

The preparation of composition 2 of Example 1 of the present invention was prepared according to the following process: Approximately 85% of the total volume of purified water was added in the compounding vessel and the specified amount of citric acid and sodium citrate were added under stirring and when complete dissolution was achieved the total amount of Flecainide acetate was added under stirring and purified water was added to the fill volume as required. The final pH value of the solution was measured with a calibrated pH-meter and, if necessary, it was adjusted to pH value according to Table 1 by using hydrochloric acid or sodium hydroxide solution. The results of compositions 1-3 of Example 1 are presented below (see Table 3).

TABLE 2
Composition 1-8 of the present invention.

| | Composition 6 | Composition 7 | Composition 8 |
|---|---|---|---|
| | Concentration | Concentration | Concentration |
| Ingredient | (mg/mL) | (mg/mL) | (mg/mL) |
| Flecainide acetate | 5.00 | 5.00 | 5.00 |
| Sodium Dihydrogen | 2.00 | — | — |
| Phosphate Dihydrate | | | |
| Di-Sodium Hydrogen | 2.50 | — | — |
| Phosphate Anhydrous | | | |
| Sodium hydroxide | q.s. to pH 7.0 | q.s. to pH 7.0 | q.s. to pH 8.0 |
| 1N Solution | | | |

Unless otherwise indicated, all steps in this procedure were carried out at room temperature.

The preparation of compositions 7 and 8 of Example 1 of the present invention was prepared according to the same manufacturing process of compositions 1 and 3 of Example 1. The preparation of composition 6 of Example 1 of the present invention was prepared according to the following manufacturing process: Approximately 85% of the total volume of purified water was added in the compounding vessel and the specified amount of Sodium Dihydrogen Phosphate Dihydrate and Di-Sodium Hydrogen Phosphate Anhydrous were added under stirring and when complete dissolution was achieved, the total amount of Flecainide acetate was added under stirring and purified water was added to the fill volume as required. The final pH value of the solution was measured with a calibrated pH-meter and, if necessary, it was adjusted to pH value according to Table 2 by using hydrochloric acid or sodium hydroxide solution.

In compositions 6 and 8 of Example 8 extensive precipitation was observed and thus no analytical activity took place. The results of composition 7 of Example 1 are presented below (see Table 3).

TABLE 3
Stability results of Compositions 1-3 and
composition 7 of Example 1 at T = 0 days

| | Composition | | | |
|---|---|---|---|---|
| | Composition | Composition | Composition | Composition |
| | 1 | 2 | 3 | 7 |
| pH | 3.67 | 3.65 | 4.17 | 7.09 |
| Appearance | Complies | Complies | Complies | Complies |
| Flecainide | 101.0 | 101.8 | 103.5 | 101.4 |
| Assay ((%) | | | | |
| Total related | 0.01 | 0.01 | 0.01 | 0.01 |
| substances of | | | | |
| Flecainide (%) | | | | |

Complies means clear solution, essentially free from visible particle

TABLE 4
Stability results of Compositions 1-3 and 7 of Example 1, at long-term
conditions (T = 30 days) (55 ± 2° C., 20 ± 5% relative humidity)

| | Composition | | | |
|---|---|---|---|---|
| | Composition | Composition | Composition | Composition |
| | 1 | 2 | 3 | 7 |
| pH | 3.73 | 3.65 | 4.22 | 7.62 |
| Appearance | Complies | Complies | Complies | Complies |
| Flecainide | — | — | — | — |
| Assay (%) | | | | |
| Total related | 0.02 | 0.02 | 0.02 | 0.11 |
| substances of | | | | |
| Flecainide (%) | | | | |

Complies means clear solution, essentially free from visible particle

TABLE 5
Stability results of Compositions 1-3 and 7 of Example 1, at long-term
conditions (T = 30 days) (40 ± 2° C., 75 ± 5% relative humidity)

| | Composition | | | |
|---|---|---|---|---|
| | Composition | Composition | Composition | Composition |
| | 1 | 2 | 3 | 7 |
| pH | 3.73 | 3.65 | 4.22 | 7.62 |
| Appearance | Complies | Complies | Complies | Complies |
| Flecainide | — | — | — | — |
| Assay (%) | | | | |
| Total related | 0.02 | 0.02 | 0.02 | 0.02 |
| substances of | | | | |
| Flecainide (%) | | | | |

Complies means clear solution, essentially free from visible particle. Upon monitoring, composition 1-3 at pH value below 4.0 was clear throughout the observation time period T=0 days to T=30 days, whereas both liquid compositions 6 and 8 of Example 1 at pH value above 7.0 was observed to exhibit a precipitate soon after preparation at time T=0 days. The chemical quality of flecainide acetate as a function of pH remained unaffected. The levels of degradants arisen upon exposure to severe heat stresses were negligible at all pH regions, demonstrating the minimal interaction of the pH factor with the reactivity of the moiety. On the contrary, the pH and potentially the type of the buffering system appeared to have an impact on the physical stability. The latter was evidenced in the form of precipitation that took place instantly in both composition 6 of Example 1, wherein phosphate compounds were added, and Composition 8 of Example 1, wherein a saturated solution occurred.

Though the pH variable was not associated with chemical instability, its potential to interact with the dissolution process of flecainide acetate strengthened the necessity for optimizing the pH of the solution as well as the buffering agents. In the absence of significant advantage of one pH region over the other, the acidic area was selected for further monitoring. This choice was substantiated by the fact that it represents a favourable substrate for the majority of the preservatives to exhibit their antimicrobial properties.

Upon standardizing the optimal pH region, a series of compatibility studies were executed, challenging the stability performance of flecainide acetate with various excipients. Representatives from the most meaningful excipients' categories, customarily encountered in oral liquid forms were subjected to evaluation: co-solvents (i.e., propylene glycol, sorbitol, glycerol), preservatives (i.e., sodium benzoate and parabens) and sweeteners (i.e., sucralose, sodium saccharin). Binary aqueous solutions of flecainide acetate with the candidate excipients were incubated at accelerated ICH conditions and elevated temperature for a time period of one month. In the context of the same study, the propensity of the selected excipients to interfere with the dissolution kinetics of the active ingredient was monitored by placing the samples at conditions able to drive supersaturation, namely cooling (at Temperature 2-8° C.).

### Example 2—Compositions 9-16 Containing 0.5% Flecainide Acetate with Various Excipients were Manufactured to Investigate their Impact on the Formation of the Precipitate

TABLE 6
Compositions 9-16 of Example 2 of the present invention

| | Composition | | | | | | | |
|---|---|---|---|---|---|---|---|---|
| | Compo | Compo | Compo | Compo | Compo | Compo | Compo | Compo |
| | 9 | 10 | 11 | 12 | 13 | 14 | 15 | 16 |
| Ingredients | Concentration (mg/mL) | | | | | | | |
| Flecainide | 5.0 | 5.0 | 5.0 | 5.0 | 5.0 | 5.0 | 5.0 | 5.0 |
| Acetate | | | | | | | | |
| Propylene Glycol | 200.0 | — | — | — | — | — | — | — |
| Glycerol | — | 200.0 | — | — | — | — | — | — |
| Sorbitol N.C. | — | — | 200.0 | — | — | — | — | — |
| Solution 70% | | | | | | | | |
| Sucralose | — | — | — | 2.0 | — | — | — | — |
| Sodium Saccharin | — | — | — | — | 2.0 | — | — | — |
| Sodium Benzoate | — | — | — | — | — | 1.0 | — | — |
| Methylparaben | — | — | — | — | — | — | 1.0 | — |
| Sodium | | | | | | | | |
| Propylparaben | — | — | — | — | — | — | — | 0.2 |
| Sodium | | | | | | | | |

| | |
|---|---|
| HCL sol 1N | q.s. to pH 3.5 |
| Purified Water | q.s. to 1.0 ml |

Compositions 9-16 of Example 2 of the present invention were prepared according to the following manufacturing process: Purified Water about 85% of the total volume was added in the compounding vessel and the total amount of the various excipients (propylene glycol, glycerol, sorbitol, sucralose, sodium saccharin, sodium benzoate, methylparaben sodium, propylparaben sodium, etc.) were added under stirring until complete dissolution. Subsequently, flecainide acetate was added under stirring until complete dissolution and purified water was added to the fill volume as required. The final pH of the solution was measured and, if required, it was adjusted to pH value approximately below 4.0, particularly to pH value about 3.5 by using hydrochloric acid solution. The generated results of said compositions are provided in the tables below.

TABLE 7
Stability results of Compositions 9-16 of Example 2 at T = 0 days

| | | | | Total related |
|---|---|---|---|---|
| | | Flecainide | | substances of |
| Composition | Appearance | Assay (%) | pH | Flecainide (%) |
| Composition 9 | Complies | 102.3 | 3.61 | B.R.L. |
| Composition 10 | Complies | 101.7 | 3.64 | B.R.L. |
| Composition 11 | Complies | 101.6 | 3.70 | B.R.L. |
| Composition 12 | Complies | N.P. | 3.69 | B.R.L. |
| Composition 13 | Complies | N.P. | 3.63 | B.R.L. |
| Composition 14 | Complies | N.P. | 3.65 | B.R.L. |
| Composition 15 | Complies | 102.2 | 3.62 | B.R.L. |
| Composition 16 | Complies | N.P. | 3.61 | B.R.L. |

N.P. states for Not Performed; B.R.L. states for Below Reporting Limit; Complies means clear solution, essentially free from visible particles.

TABLE 8
Stability results of Compositions 9-16 of Example
2 at long-term conditions (storage period T =
30 days) (55 ± 2° C., 20 ± 5% relative humidity)

| | | | Total related |
|---|---|---|---|
| | | | substances of |
| Composition | Appearance | pH | Flecainide (%) |
| Composition 9 | Presence of particles | 3.72 | — |
| Composition 10 | Presence of particles | 3.70 | — |
| Composition 11 | Clear solution | 3.69 | 0.02 |
| Composition 12 | Clear solution | 3.72 | 0.02 |
| Composition 13 | Clear solution | 3.73 | 0.01 |
| Composition 14 | Clear solution | 3.68 | 0.02 |
| Composition 15 | Presence of crystals | 3.69 | — |
| Composition 16 | Presence of crystals | 3.68 | — |

Due to precipitation observed upon 30 days storage, compositions 9, 10, 15 and 16 of Example 2 were not chemically examined.

TABLE 9
Stability results of Compositions 9-16 of Example 2
at long-term conditions (storage period T = 30
days) (Temperature 40 ± 2° C., 75 ± 5% relative humidity)

| | | | Total related |
|---|---|---|---|
| | | | substances of |
| Composition | Appearance | pH | Flecainide (%) |
| Composition 9 | Presence of crystals | 3.68 | — |
| Composition 10 | Presence of crystals | 3.66 | — |
| Composition 11 | Clear solution | 3.67 | 0.02 |
| Composition 12 | Clear solution | 3.72 | 0.02 |
| Composition 13 | Clear solution | 3.64 | 0.02 |
| Composition 14 | Clear solution | 3.75 | N.D. |
| Composition 15 | Presence of crystals | 3.66 | — |
| Composition 16 | Presence of crystals | 3.74 | — |

N.D. states for Not Detected

TABLE 10
Stability results of Compositions 9-16 of Example 2 at storage conditions
T = 30 days storage period, at temperature 2-8° C.

| | | Flecainide |
|---|---|---|
| Composition | Appearance | Assay (%) |
| Composition 9 | Precipitate in the form of crystals | 62.5 |
| Composition 10 | Precipitate in the form of crystals | 73.0 |
| Composition 11 | Presence of dispersed particles | 100.6 |
| Composition 12 | Clear solution | — |
| Composition 13 | Clear solution | — |
| Composition 14 | Clear solution | — |
| Composition 15 | Precipitate in the form of crystals | 60.7 |
| Composition 16 | Precipitate in the form of crystals | — |

All compatibility studies demonstrated thermal stability from a chemical perspective, as manifested by the extremely low level of degradants. Conversely, physical destabilization in the form of precipitation occurred with certain excipients. Namely, the presence of propylene glycol and glycerol suppressed the solubility of flecainide acetate forcing it to salt out. It is of interest that alerting findings of instability were already obtained from the favourable for solubilization effect of heat, rendering the intensification of this phenomenon in refrigerating conditions well-anticipated. It is postulated that the reduction of the dielectric constant of water mediated by the addition of propylene glycol and glycerol minimized the solubilization capacity of the medium resulting in crystallization of the active ingredient. Said hypothesis was analytically verified through the conduct of assay tests in samples containing visible matter. Significant potency losses were recorded, well correlated with the output of the visual inspection. Thus, the incorporation of organic solvents in the liquid vehicle was ruled out.

A similar effect was noted for methyl- and propylparaben eliminating them from the rest of formulation development studies.

Overall, it became apparent that formulating flecainide acetate in solution requires a lean aqueous platform comprising the minimum necessary components that impart the desired state of quality and acceptability. The following components were found appropriate for further evaluation: sucralose, sodium saccharin, sodium benzoate, sorbitol.

### Example 3: Evaluation/Selection of the Buffering Agents

### Example 3—Compositions 4a, 4b and 4c Containing 0.5% Flecainide Acetate and Various Buffering Agents

Details on the compositions and their stability results are shown in the following tables.

TABLE 11
Compositions 4a, 4b and 4c of Example 3 of the present invention.

| | Composition 4a | Composition 4b | Composition 4c |
|---|---|---|---|
| | Concentration | Concentration | Concentration |
| Ingredients | (mg/mL) | (mg/mL) | (mg/mL) |
| Flecainide | 5.00 | 5.00 | 5.00 |
| acetate | | | |
| Acetic acid 30% | q.s. to pH 3.0 | — | — |
| solution | | | |
| HCl 1N | — | q.s. to pH 3.0 | — |
| Solution | | | |
| Citric acid 30% | — | — | q.s. to pH 3.0 |
| solution | | | |
| Purified Water | q.s. to 1.0 mL | q.s. to 1.0 mL | q.s. to 1.0 mL |

Guided by the compatibility studies, the type of the buffering system was identified as a key variable for the chemical and physical stability of the drug product. Thus, composition with various buffering systems were evaluated, and the stability performance of said compositions, and mainly the physical one, was assessed.

To conclusively understand the potential interference of the buffering components on the dissolution process of the active ingredient itself, conditions able to stress the solubility property were employed. This meant that normal conditions (at temperature of 25° C.±2° C./and relative humidity 60%±5%) and refrigerating conditions (5° C.±3° C.) storage was implemented for one month. Appearance, assay and impurities before and after storage were determined.

Compositions 4a, 4b and 4c of Example 3 of the present invention was prepared according to the same manufacturing process of composition 2 of Example 1 of the present invention and the pH value was adjusted to approximately 3.0 with the addition of either acetic acid 30% solution, hydrochloric acid solution or citric acid 30% solution.

The bottles of Compositions 4a, 4b and 4c of Example 3 were exposed at various storage conditions and their stability results are shown in Table 12 below. The stability results of Compositions 4a and 4c of Example 3 over 1 month showed that said compositions of Flecainide acetate comprising citric acid or acetic acid as an acidifying agent at final pH value of 3.0 is also stable and impart an acidic pH environment in the dosage form and protect the active ingredient from degradation (see Table 12).

TABLE 12
Stability results of Compositions 4a, 4b and
4c of Example 3 at various storage conditions

| | | | |
|---|---|---|---|
| | | | Total related |
| | Flecainide | | substances of |
| Appearance | Assay (%) | pH | Flecainide (%) |

at storage period T = 0 days

| | | | | |
|---|---|---|---|---|
| Composition 4a | Clear solution | 97.3 | 3.01 | N.D |
| Composition 4b | Clear solution | 98.6 | 3.00 | N.D |
| Composition 4c | Clear solution | 98.5 | 3.11 | N.D |

at long-term conditions: storage period T = 30 days,
at temperature 25 ± 2° C., 60 ± 5% relative humidity)

| | | | | |
|---|---|---|---|---|
| Composition 4a | Clear solution | 97.2 | 3.04 | 0.01 |
| Composition 4b | Precipitate, | N.P. | N.P. | N.P |
| | crystals | | | |
| Composition 4c | Clear solution | 98.3 | 3.10 | 0.01 |

at storage conditions: storage period T = 30 days,
at temperature 5° C. ± 3° C.

| | | | | |
|---|---|---|---|---|
| Composition 4a | Clear solution | 97.2 | 3.04 | 0.01 |
| Composition 4b | Precipitate, | N.P. | N.P. | N.P. |
| | crystals | | | |
| Composition 4c | Clear solution | 98.2 | 3.10 | 0.01 |

N.D. states for Not Detected;
N.P. states for Not Performed

Visual observations confirmed the dependency of the physical stability of the solution of flecainide acetate with the type of the buffering system. Precipitation occurred in composition 4b of Example 3 in the presence of hydrochloric acid; a behaviour that was intensified upon refrigerating storage. No such solid structures were detected in compositions 4a and 4c of Example 3 with the organic acids under evaluation. Both acetic and citric acids did not hinder the dissolution process of the active ingredient, even after long-term exposure to conditions able to induce crystal growth. The accumulated results were in good agreement with the descriptions provided in the compendial monographs. With the generated data affording a deeper understanding of the interactions between Flecainide acetate and acidifying agents, the selection of acetic or citric acids for mediating said pH value was reasonable.

At this point, it should be clarified that the data referring to HCl-containing compositions in this study do not contradict the ones produced during the compatibility study. The reason for not mentioning precipitation in the pH stability study with the HCl-acidified compositions is attributed to the high energy input introduced by the heat that increased the mobility of the solute molecules facilitating their passage in solution.

Finally, the citric acid was chosen for the most preferred composition of the present invention. The very strong acidic odour of acetic acid, unlikely to be effectively concealed by formulation agents, directed the selection of citric acid at the benefit of acceptability. Its level was fixed at 6.85 mg/ml which equates to the quantity required for bringing the pH value to the preferred target 2.8-3.2.

### Example 4—Composition 5 Containing 0.5% Flecainide Acetate (Reference Example)

TABLE 13
Composition 5 of the present invention containing
Flecainide acetate 25 mg/5 mL Oral Solution

| | Composition 5 | |
|---|---|---|
| Ingredient | Quantity [mg] | Function |
| Flecainide acetate | 5.00 | Active ingredient |
| Sodium benzoate | 0.80 | Preservative |
| Sucralose | 1.20 | Sweetening agent |
| Cherry Flavour | 0.50 | Flavouring agent |
| Citric acid | 6.85 | Acidifying agent |
| Citric acid | q.s. to pH 2.8-3.2 | pH adjusting agent |
| Purified Water | q.s. 1.0 mL | Solvent/Diluent |

Unless otherwise indicated, all steps in this procedure were carried out at room temperature.

Composition 5 of Example 4 of the present invention which is the most preferred composition according to the present invention was prepared according to the following manufacturing process: Approximately 85% of the total volume of purified water was added in the compounding tank and then the specified amount of citric acid was added under stirring until complete dissolution at water temperature from 23-28° C. In the obtained mixture the total quantity of sodium benzoate was charged under stirring until complete dissolution. Subsequently, Flecainide acetate was added under stirring until complete dissolution and sequentially the quantities of sucralose and cherry flavour were added under continuous stirring. A sample of the solution was obtained to measure the pH value using a calibrated pH-meter, and if required, the pH value of the final solution was adjusted to pH values from 2.8 to 3.2 with the aid of citric acid 30% w/v solution. Then, the remaining amount of purified water was added to the final volume under stirring as required.

The final solution was then passed through a polypropylene (PP) cartridge filter of 30 or 40 μm porosity and filled into the designated bottles for oral use.

In order to complete the characterization of the composition of the present invention, the level of preservative was assessed against its antimicrobial protection by means of Preservative Efficacy Test (PET) and the level of sucralose and the type of flavour were assessed by a sensory panel in terms of taste and aftertaste (palatability studies).

### Example 5: Microbiological Evaluation of the Preservative

### Example 5—Compositions 5a, 5b, 5c and 5d Containing 0.5% Flecainide Acetate and Various Concentrations of Sodium Benzoate

Details on the compositions and their stability results are shown in the following tables

TABLE 14
Compositions 5a, 5b, 5c and 5d of the present
invention subjected to PET studies

| | | | |
|---|---|---|---|
| Compo- | Compo- | Compo- | Compo- |
| sition | sition | sition | sition |
| 5a | 5b | 5c | 5d |

| | |
|---|---|
| Ingredient | Concentration [mg/mL] |

| | | | | |
|---|---|---|---|---|
| Flecainide | 5.0 | 5.0 | 5.0 | 5.0 |
| Acetate | | | | |
| Sucralose | 1.2 | 1.2 | 1.2 | 1.2 |
| Cherry Flavour | 0.5 | 0.5 | 0.5 | 0.5 |
| Citric Acid | 6.85 | 6.85 | 6.85 | 6.85 |
| Anhydrous | | | | |
| Sodium Benzoate | 0.8 | 1.0 | 1.2 | 0.6 |
| Citric acid 30% | q.s to | q.s. to | q.s. to | q.s. to |
| | pH 3.0 | pH 3.0 | pH 3.0 | pH 3.0 |

| | |
|---|---|
| Purified Water | q.s. to 1.0 mL |

Compositions 5a, 5b, 5c and 5d of Example 5 were also tested for microbiological stability with various concentrations of sodium benzoate, wherein solutions of said compositions 5a, 5b, 5c and 5d were stored in amber glass bottles at room temperature. The stability was evaluated on days 0, 14 and 28 in both “after opening” and “before opening” conditions for the composition containing the preservative and used as a control. Bottles were opened in non-sterile conditions. Each analysis was performed in duplicate.

Microbiological analyses were performed according to the European Pharmacopeia monograph for non-sterile products, using the Preservative Efficacy Test (PET). European Pharmacopeia requirements indicate a total aerobic microbial count (TAMC) of less than 10² CFU/mL, a total yeast and moulds count (TYMC) of less than 10¹ CFU/mL, and the absence of Escherichia coli.

The preservative efficacy test results of all compositions 5a, 5b, 5c and 5d tested were satisfactory. A detailed presentation of the biocidal rates exhibited by the different levels of sodium benzoate is given in the below tables.

TABLE 15
Preservative efficacy test of composition 5a of the present invention

| | Units in cfu/g | | | | Log Reduction | |
|---|---|---|---|---|---|---|
| Parameter | Inoculation | T = 0 | 14^{th} Day | 28^{th} Day | 14^{th} Day | 28^{th} Day |
| Escherichia coli | 5.2E+05 | 1.6E+04 | <10 | <10 | 4.88 | 4.88 |
| Pseudomonas aeruginosa | 5.4E+05 | 5.0E+04 | <10 | <10 | 4.72 | 4.72 |
| Staphylococcus aureus | 7.6E+05 | 7.1E+05 | <10 | <10 | 4.73 | 4.73 |
| Candida albicans | 4.2E+05 | 3.8E+05 | <10 | <10 | 4.62 | 4.62 |
| Aspergillus brasiliensis | 2.2E+05 | 2.1E+05 | <10 | <10 | 4.34 | 4.34 |

TABLE 16
Preservative efficacy test of composition 5b of the present invention

| | Units in cfu/g | | | | Log Reduction | |
|---|---|---|---|---|---|---|
| Parameter | Inoculation | T = 0 | 14^{th} Day | 28^{th} Day | 14^{th} Day | 28^{th} Day |
| Escherichia coli | 5.2E+05 | 4.0E+03 | <10 | <10 | 4.88 | 4.88 |
| Pseudomonas aeruginosa | 5.4E+05 | 1.7E+04 | <10 | <10 | 4.72 | 4.72 |
| Staphylococcus aureus | 7.6E+05 | 7.2E+05 | <10 | <10 | 4.73 | 4.73 |
| Candida albicans | 4.2E+05 | 3.8E+05 | <10 | <10 | 4.62 | 4.62 |
| Aspergillus brasiliensis | 2.2E+05 | 2.6E+05 | <10 | <10 | 4.34 | 4.34 |

TABLE 17
Preservative efficacy test of composition 5c of the present invention

| | Units in cfu/g | | | | Log Reduction | |
|---|---|---|---|---|---|---|
| Parameter | Inoculation | T = 0 | 14^{th} Day | 28^{th} Day | 14^{th} Day | 28^{th} Day |
| Escherichia coli | 5.2E+05 | 2.0E+03 | <10 | <10 | 4.88 | 4.88 |
| Pseudomonas aeruginosa | 5.4E+05 | 7.0E+03 | <10 | <10 | 4.72 | 4.72 |
| Staphylococcus aureus | 7.6E+05 | 7.1E+05 | <10 | <10 | 4.73 | 4.73 |
| Candida albicans | 4.2E+05 | 4.2E+05 | <10 | <10 | 4.62 | 4.62 |
| Aspergillus brasiliensis | 2.2E+05 | 1.9E+05 | <10 | <10 | 4.34 | 4.34 |

TABLE 18
Preservative efficacy test of Composition 5d of the present invention

| | Units in cfu/g | | | | Log Reduction | |
|---|---|---|---|---|---|---|
| Parameter | Inoculation | T = 0 | 14^{th} Day | 28^{th} Day | 14^{th} Day | 28^{th} Day |
| Escherichia coli | 4.5E+05 | 5.3E+04 | <10 | <10 | 4.60 | 4.60 |
| Pseudomonas aeruginosa | 1.7E+05 | 9.5E+04 | <10 | <10 | 4.65 | 4.65 |
| Staphylococcus aureus | 4.0E+05 | 9.5E+04 | <10 | <10 | 4.23 | 4.23 |
| Candida albicans | 3.4E+05 | 1.4E+05 | 2.7E+01 | <10 | 4.10 | 4.53 |
| Aspergillus brasiliensis | 1.5E+05 | 1.2E+05 | 1.2E+03 | 2.5E+02 | 2.10 | 2.79 |

According to the generated results, all PET studies complied well with the Ph. Eur. Standards for preservative efficacy set forth in § 5.1.3 “Efficacy of antimicrobial preservation”. In particularly, the number of bacteria and yeast/fungi recovered per gram was easily reduced by a log reduction higher than 3 and 1 respectively while no increase observed on the 28^{th} day.

The employed range of sodium benzoate from 0.6 to 1.2 mg/ml exhibited adequate biocidal effect against bacteria-either gram positive or gram negative, while a concentration dependency was noted for its antifungal capacity. The latter was reflected in the microbial quality of the composition 5d containing the lowest level of preservative (0.6 mg/ml) whereby comparatively higher microbial counts (expressed by lower logarithmic reduction) of Aspergillus brasiliensis were recorded. Although the aforementioned decline fulfilled satisfactorily the preservation criteria, it was translated to higher sensitivity of the developed carrier to this pathogen.

### Example 6: Palatability Studies and Evaluation of Sucralose and Flavouring Agents

TABLE 19
Example 6 - Compositions 5e, 5f, 5g and 5h of the present invention
containing 0.5% Flecainide acetate subjected to Palatability studies

| | | | |
|---|---|---|---|
| Compo- | Compo- | Compo- | Compo- |
| sition | sition | sition | sition |
| 5e | 5f | 5g | 5h |

| | |
|---|---|
| Ingredient | Concentration [mg/mL] |

| | | | | |
|---|---|---|---|---|
| Flecainide | 5.0 | 5.0 | 5.0 | 5.0 |
| Acetate | | | | |
| Sodium Benzoate | 0.5 | 0.5 | 0.5 | 0.5 |
| Citric Acid | 6.85 | 6.85 | 6.85 | 6.85 |
| Sucralose | 1.0 | 1.2 | 1.2 | 1.2 |
| Strawberry Flavour | X | X | X | 0.5 |
| Cherry Flavour | X | 0.5 | X | X |
| Orange Flavour | X | X | 0.5 | X |
| Citric acid 30% | q.s to | q.s. to | q.s. to | q.s. to |
| solution | pH 3.0 | pH 3.0 | pH 3.0 | pH 3.0 |

| | |
|---|---|
| Purified Water | q.s. to 1.0 mL |

TABLE 20
Palatability responses to Compositions 5e,
5f, 5g and 5h of the present invention

| | | Compo- | Compo- | Compo- | Compo- |
|---|---|---|---|---|---|
| | | sition | sition | sition | sition |
| Taste Qualities | | 5e | 5f | 5g | 5h |
| Subject | Taste | 3 | 4 | 3 | 3 |
| # 1 | Aftertaste | 3 | 4 | 3 | 3 |
| Subject | Taste | 3 | 4 | 2 | 3 |
| # 2 | Aftertaste | 4 | 3 | 2 | 3 |
| Subject | Taste | 3 | 4 | 2 | 2 |
| # 3 | Aftertaste | 3 | 3 | 2 | 3 |
| Subject | Taste | 3 | 5 | 2 | 3 |
| # 4 | Aftertaste | 3 | 4 | 2 | 3 |
| Subject | Taste | 3 | 4 | 2 | 3 |
| # 5 | Aftertaste | 3 | 3 | 2 | 2 |

| | | | | |
|---|---|---|---|---|
| Total | 31 | 38 | 22 | 28 |

In accordance with the verbal judgement of the participants, the main taste quality that required amelioration was that of sour taste. The highest score was assigned to composition 5f that comprised sucralose at 1.2 mg/ml and cherry flavour at 0.5 mg/ml. As per the participants' responses, composition 5e and composition 5f were sufficiently palatable with the latter taking the lead due to its additive effect on taste and odour. Deterioration of the organoleptic profile was reported when cherry flavour was replaced by orange and strawberry agents in compositions 5g and 5h. These flavours accentuated the sourness of the preparation, rationalizing their exclusion from the final formulation.

While the present invention has been described with respect to the particular embodiments, it will be apparent to those skilled in the art that various changes and modifications may be made in the invention without departing from the spirit and scope thereof, as defined in the appended claims.

1. A storage-stable liquid pharmaceutical composition for oral administration comprising a therapeutically effective amount of flecainide or a pharmaceutically acceptable salt thereof, as an active ingredient, wherein said active ingredient is from about 0.1 to about 3.0% w/v, wherein the weight/volume percentages are expressed as g/100 mL units, wherein said composition has a pH value below 4.0.

2. The liquid composition according to claim 1, wherein said active ingredient is preferably from about 0.4 to about 1.0% w/v, and more preferably about 0.5% w/v.

3. The liquid composition according to claim 1, wherein said pH value is preferably in the range from 2.8 to 3.2, and more preferably 3.0.

4. The liquid composition according to claim 1, wherein Flecainide is in the form of acetate salt.

5. The liquid composition according to claim 4, wherein said composition further comprises at least one of the acceptable excipients selected from sweeteners, buffering agents, preservatives, pH adjusting agents, solubilizers, flavouring agents or mixtures thereof.

6. The liquid composition according to claim 4, wherein said composition comprises a citrate buffering system such as acetic acid, citric acid or mixtures thereof.

7. The liquid composition according to claim 4, wherein said composition comprises sucralose as a sweetener.

8. The liquid composition according to claim 4, wherein said composition comprises cherry flavour as flavouring agent in the range of 0.5 mg/mL.

9. The liquid composition according to claim 4, wherein said composition comprises sodium benzoate as preservative.

10. The liquid pharmaceutical composition according to claim 9, wherein said sodium benzoate is in the range from 0.6 to 1.2 mg/mL.

11. A storage-stable liquid composition comprising 0.5% Flecainide acetate, sucralose, a citrate buffering system which comprises citric acid, acetic acid or mixtures thereof, sodium benzoate in the range from 0.6 to 1.2 mg/mL and said composition is having pH value in the range from 2.8 to 3.2.

12. A process for the preparation of a storage-stable liquid composition for oral administration comprising a therapeutically effective amount of flecainide or a pharmaceutically acceptable salt thereof, as an active ingredient, wherein said active ingredient is from about 0.1 to about 3.0% w/v wherein the weight/volume percentages are expressed as g/100 mL units, wherein said process comprises the following steps:
(a) dissolving in purified water at least one of the excipients selected from citrate buffering system, and/or preservatives;
(b) adding the total amount of flecainide or salt thereof, to the resulting solution of step (a) and stirring for appropriate time until complete dissolution;
(c) optionally adding to the solution of step (b) at least one of the excipients selected from sweeteners and/or flavouring agents; and
(d) adjusting the pH of the final solution to a value below 4.0 with suitable citric acid solution, or hydrochloric acid or sodium hydroxide solution.

13. The process for the preparation of a composition according to claim 12, wherein further comprises a step of filtering the solution through a cartridge filter, which preferably has a nominal porosity of 30 or 40 μm.

14. The process for the preparation of a composition according to claim 12, wherein in step (a) sodium benzoate is added as preservative.

15. The process for the preparation of a composition according to claim 12, wherein in step (c) sucralose is added as sweetener and cherry flavour is added as flavouring agent.
